(19) United States
(12) Patent Application Publication
(10) Pub. No.: US 2015/0245788 A1
(43) Pub. Date: Sep. 3, 2015

(54) COUGH DETECTION, ANALYSIS, AND COMMUNICATION PLATFORM

(71) Applicants: Quvium UK Ltd; Parilogix, LLC, Woburn, MA (US)

(72) Inventor: Steven P. Schmidt, Salem, NH (US)

(21) Appl. No.: 14/698,926

(22) Filed: Apr. 29, 2015

Related U.S. Application Data
(63) Continuation-in-part of application No. 14/336,180, filed on Jul. 21, 2014.
(60) Provisional application No. 61/856,803, filed on Jul. 22, 2013.

(30) Foreign Application Priority Data
Mar. 26, 2015 (GB) 1505214.5

(51) Int. Cl. A61B 5/08; A61B 7/04; A61B 5/00

(52) U.S. Cl. CPC A61B 5/0823; A61B 5/0022; A61B 7/04; A61B 5/7275

(57) ABSTRACT

A cough detection, analysis, and communication platform may be provided. The platform may comprise methods, systems, and devices for detecting, analyzing, and communicating cough detection data. A cough detection device may comprise at least one microphone and analytic firmware used to employ methods of detecting, analyzing, and communicating the cough detection data. The analytic software may be updated to use the appropriate algorithms for an individual or corresponding demographic based on, for example, age and disease, with abnormal cough frequency components, abnormal cough frequency profiles, and alert thresholds. The cough detection device may be configured to communicate cough detection data to a server. The server, in turn, may be operative to process and communicate the data to a third party, such as, for example, a physician. Systems may further be operative to provide a user interface (e.g., mobile or web applications) for interfacing with the platform.

### PRIORITY CLAIMS

The present application is a continuation-in-part of earlier filed U.S. Utility patent application Ser. No. 14/336,180 filed on Jul. 21, 2014 to Quvium UK Ltd, the Applicant of the present application. The disclosures of each of the referenced applications is hereby incorporated herein.

Furthermore, U.S. Utility patent application Ser. No. 14/336,180 claims the benefit of U.S. Provisional Patent Application No. 61/856,803, entitled “Cough Detection,” invented by Steven P. Schmidt, and filed to the United States Patent and Trademark Office on Jul. 22, 2013. The disclosure of the provisional application is incorporated herein by reference.

The present application further claims priority to U.K. Utility Patent Application No. 1505214.5, filed on Mar. 26, 2015 to Quvium UK Ltd, the Applicant of the present application.

### RELATED APPLICATIONS

Related U.K. Utility Patent Application No. 1412909.2, filed by the same Applicant on Jul. 21, 2014 is incorporated herein.

Related U.S. patent application Ser. No. 13/011,394, filed by the same inventor and entitled “Early Warning Method and System for Chronic Disease Management,” is incorporated herein by reference.

### TECHNICAL FIELD

The technical field of the present disclosure generally relates to medical devices and remote health-care services.

### BACKGROUND

Patients who have chronic lung disease may experience regular coughing. Sudden increases in their regular coughing frequencies may be an early indicator of respiratory compromise (infection) or impending exacerbation event such as, for example, an asthma attack for people with asthma. In various cases (e.g., Cystic Fibrosis, Asthma, Chronic Obstructive Pulmonary Disease (COPD), Pulmonary Fibrosis, Bronchitis, Respiratory infection, Lung Cancer, Gastro Esophageal Reflux Disease (GERD), and other diseases affecting normal lung operation), a strong indicator of respiratory compromise is an increase in cough frequency which may be due to pathophysiology in the lungs from increasing cough sensitivity and a response to cell influx into lungs. For example, in an impending asthma attack coughing may be an increase in a patients coughing rate from a regular rate of, for example, approximately one cough per two hours to a much higher rate. This increased cough frequency may begin to occur approximately three hours before an exacerbation event (“the attack”) in normal case or, for hypersensitive individuals, as late as half an hour before the attack.

Doctor supplied treatment plans have an intervention strategy to prevent a full blown attack at the sign of worsening symptoms (e.g. the increased cough rate). However, these treatment plans may only work if the doctor or patient are aware of the worsening symptoms and act upon this in a timely manner timely act upon this.

An early warning may encourage the patient to seek early treatment. Early treatment, in turn, with rescue medication (e.g., an inhaler) or removal of the exacerbation trigger (e.g., emotional stress such as crying, air pollution, etc.) may impede the full impact of the exacerbation event. Impeding the full impact of the exacerbation event may decrease the likelihood of emergency room visits and hospitalizations for the patient.

Unfortunately some patients, such as children and the elderly, often ignore their early symptoms until early treatment measures are no longer effective, making conventional intervention strategies more likely to fail. Consequently, there is a need for effective and efficient early symptom detection devices that operate passively with minimal patient intervention or noticeability.

### BRIEF SUMMARY

This brief summary is provided to introduce a selection of concepts in a simplified form that are further described below in the Detailed Description. This brief summary is not intended to identify key features or essential features of the claimed subject matter. Nor is this brief overview intended to be used to limit the claimed subject matter's scope.

A cough detection, analysis, and communication platform may be provided. The platform may include methods, systems, and a devices for detecting, analyzing, and communicating cough detection data. A cough detection device of the platform may comprise at least one microphone and an analytic software used to employ methods of detecting, analyzing, and communicating the cough detection data.

Consistent with embodiments of the present disclosure, data collected by the cough detection device (e.g., cough frequency) may then be communicated to at least one server. The server may, in turn, be operative to process and communicate the received data to an interested party, such as, but not limited to, a physician. Having the user's cough-frequency date, the physician may then act on the received information in order to, for example, assess the user symptoms and take appropriate action.

The platform may further include a user interface (e.g., mobile or web applications) for enabling users, physicians, and other entities to interface with the platform. For example, a user may access the user interface to configure the parameters and settings of the detection device, including, but not limited to, for example, alert settings. Similarly, a physician may access the user interface in order to, for example, view the processed cough detection data and recommend, through the interface, a treatment for the user. In turn, the user may access the interface to view the physician's recommendations.

Both the foregoing brief summary and the following detailed description provide examples and are explanatory only. Accordingly, the foregoing brief summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a first embodiment of a cough detection device;

FIG. 2A illustrates a second embodiment of a cough detection device;

FIGS. 2B-2D illustrate a third embodiment of a cough detection device;

FIG. 3A-3C illustrates a fourth embodiment of a cough detection device;

FIG. 4 illustrates an operating environment for a platform consistent with embodiments of the present disclosure;

FIGS. 5A-5B illustrates flow charts of methods for providing the platform consistent with embodiments of the present disclosure;

FIGS. 6A-6B illustrate waveforms of a cough detection signal;

FIGS. 7A-7D illustrate waveforms of a processed cough detection signal using a first signal processing method;

FIGS. 8A-8B illustrate waveforms of a processed cough detection signal using a second signal processing method;

FIGS. 9A-9B illustrates architectures of a detection device consistent with embodiments of the present disclosure; and

FIG. 10 illustrates a computing device consistent with embodiments of the present disclosure.

### DETAILED DESCRIPTION

The following detailed description refers to the accompanying drawings. Wherever possible, the same reference numbers are used in the drawings and the following description to refer to the same or similar elements. While embodiments of the invention may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the invention. Instead, the proper scope of the invention is defined by the appended claims. The present disclosure contains headers. It should be understood that these headers are used as references and are not to be construed as limiting upon the subjected matter disclosed under the header.

I. Detection Device Design

Embodiments of the present disclosure may provide a cough frequency detection device operative to detect, analyze, and communicate a user's coughing frequency. The detection device may be further operative to provide an indication upon a detection of an increased coughing rate. The indication may serve as an early warning of a physiological cascade of lung problems leading to an exacerbation event. The exacerbation event may be, for example, but not be limited to, an asthma attack.

The detection device may be integrated with, by way of non-limiting example, a pendant, watch, pocket FOB, an adhesive sleeve or patch, and a zipper attached FOB. The form of the detection device may vary with considerations of a user's demographics, such as age and gender. The detection device may be small, portable, and have a substantially long battery life. In some embodiments, the detection device may be rechargeable, while in other embodiments the detection device may be disposable.

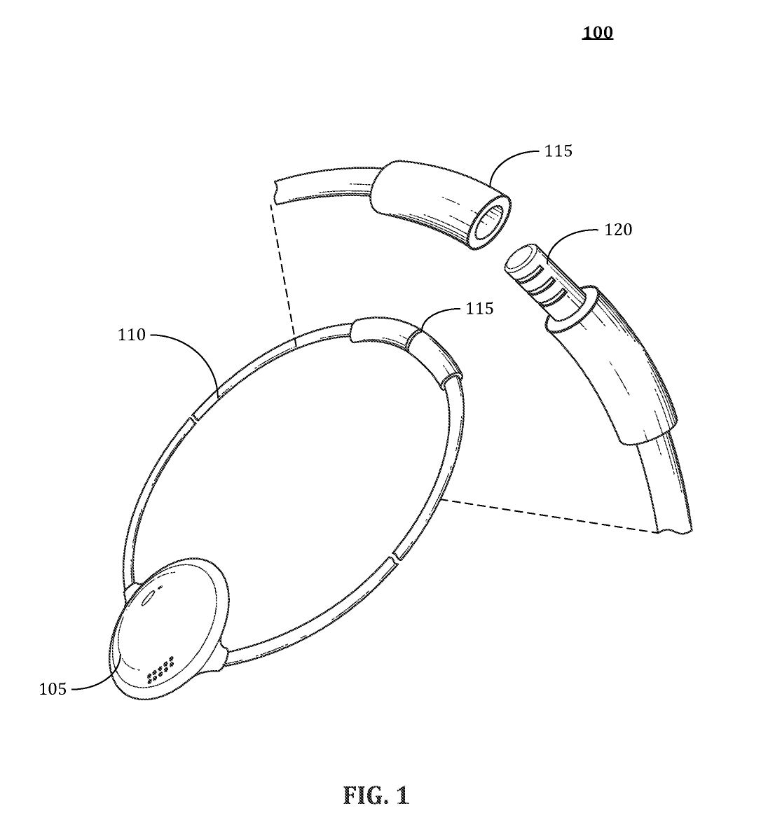

FIGS. 1-3 illustrates various embodiments of the cough detection device. As shown in FIG. 1, detection device 100 may be worn by a user around a user's neck. Detection device 100 may be integrated into various accessories that may be configured to be worn by a user.

For example, in some embodiments, the device 100 may be integrated with a housing 105, a bracing means 110 and a clasp 115. Housing 100 may be comprised of, but not limited to, for example, a rubber and/or plastic material designed to a house the device components for providing and interfacing with the methods and systems of the present disclosure. The bracing means 105 may provide a mechanism to secure device 100 to the user. The mechanism may comprise, for example, a necklace, a bracelet, a ring, or any other suitable article that may be worn by the user. The mechanism may further serve to provide an electronic interface (e.g., wiring and circuitry) from the housing 105 to the clasp 115.

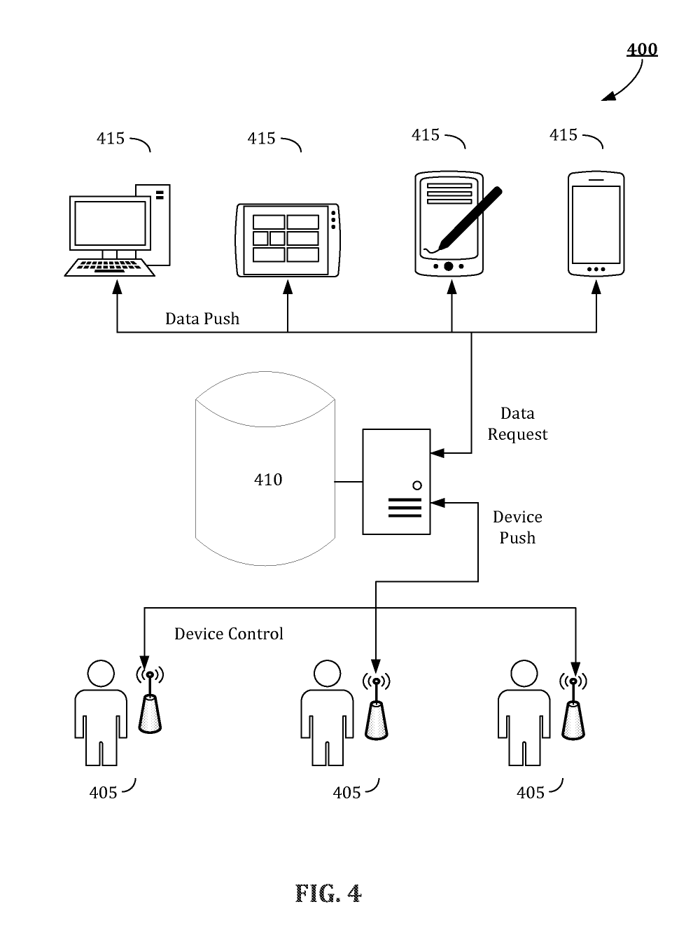

In addition to securely fastening the device to the user, clasp 115 may be configured to serve as, for example, a port 120. Port 120, in turn, may be configured to serve as a charging device or, in some embodiments a communications port (e.g., a universal serial bus (USB) port or similar computing interface). The port may provide energy to the device and/or exchange information between device 100 and an external system as will be detailed below, at least in reference to FIG. 4.

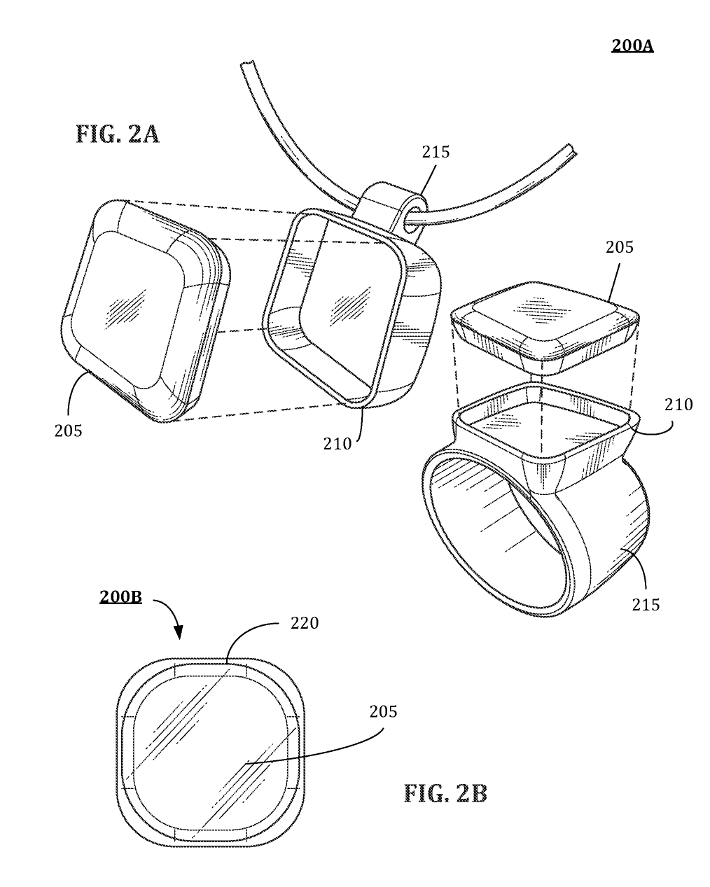

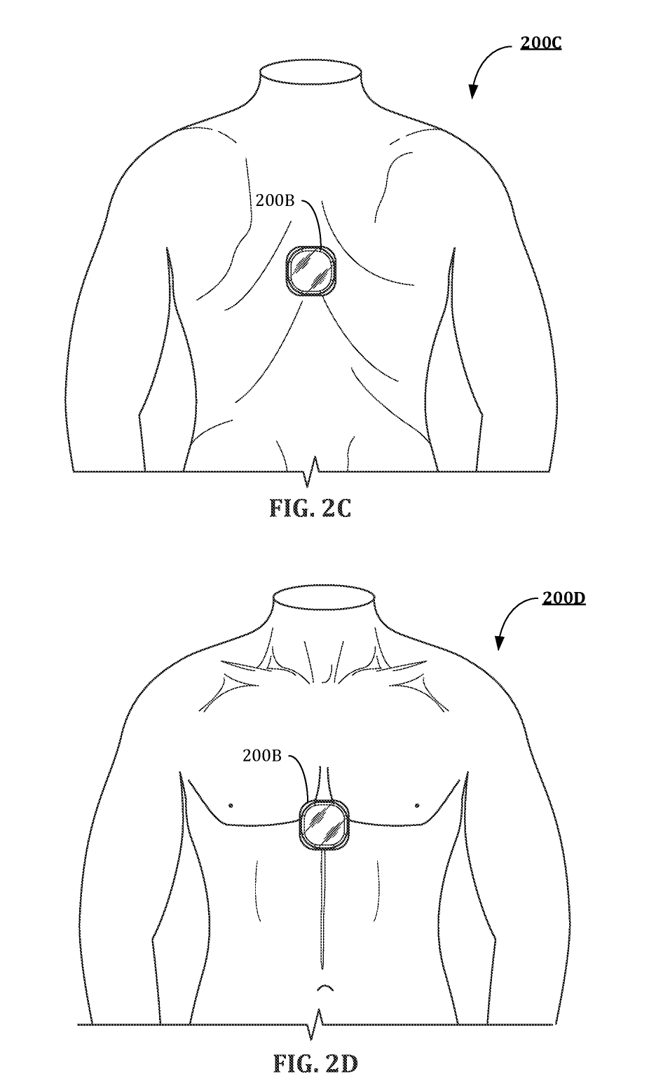

Still consistent with embodiments of the present disclosure, the detection device may be designed for insertion into different user accessories. For example, as shown in FIG. 2A, a detection device 200A may comprise a detachable module 205. Detachable module 205 may comprise a housing for the detection device components. The housing may be insertable into a socket 210. Socket 210 may be configured to attach to a bracing means 215. Bracing means 215 may be designed as, or an attachable portion of, for example, but not limited to, a necklace, a ring, or a bracelet.

FIG. 2B yet further embodiments of the present disclosure, the detection device may be designed as an adhesive sleeve or patch 200B. Patch 200B may be an adhesive material designed to adhere to, for example, a user's body (e.g., directly to the user's skin). Patch 200B may form a pocket in which detection module 205 may be placed. An antenna 220 may be wrapped around a circumference of patch 200B or detection module 205.

FIG. 2C illustrates an embodiment 200C in which patch 200B is attached to a user's back. FIG. 2D illustrates an embodiment 200D in which patch 200B is attached to a user's sternum. Such form factor of the adhesive sleeve or patch 200B may enable detection module 205 to detect sounds more precisely as well as additional sounds that may not be detectable with other form factors (e.g., breathing rates). It should be understood that embodiments 200C and 200D are examples and that the adhesive sleeve may be applied to other portions of the user's body.

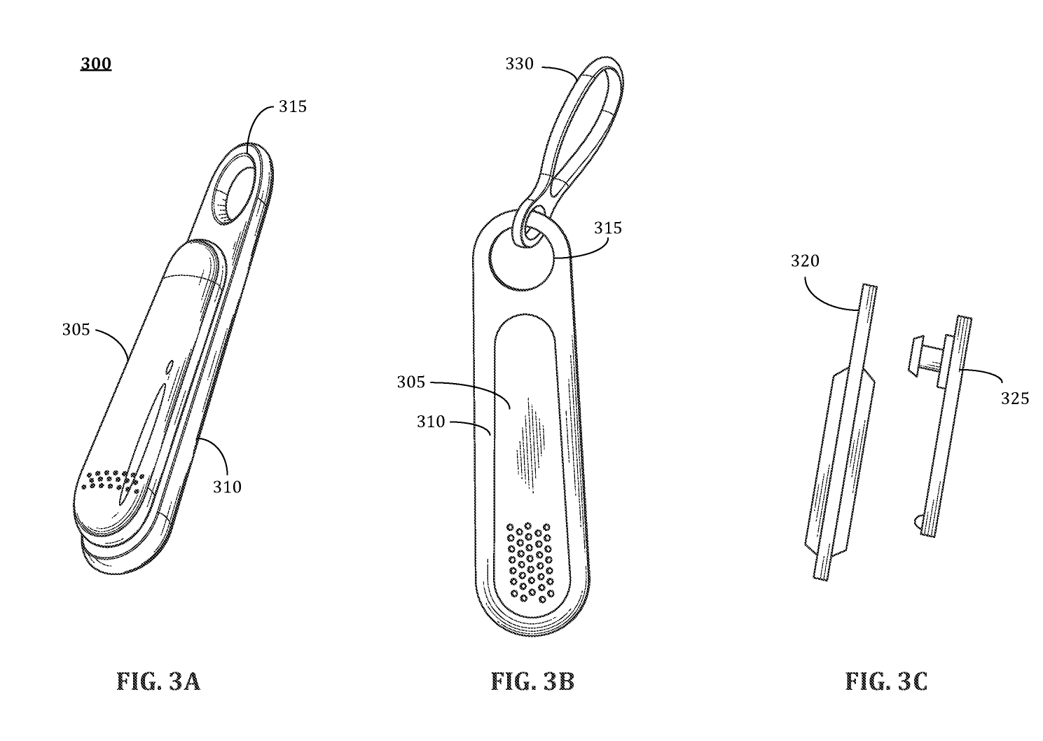

FIGS. 3A-3C illustrate additional embodiments of a detection device. Detection device 300 may be comprised of a detection module 305 having a housing for the detection device components. The housing may be configured to be inserted into a socket 310 comprised of, for example, a mounting ring 310. Mounting ring 315 may be configured to attach to, for example, a user accessory 330. In some embodiments, mounting ring 315 may comprise a port 320. Port 320 may serve as an attachment means to detection device 300. In some embodiments, the port 320 may be configured to couple detection module 300 into an electronic interface such as, for example, but not limited to a snap-in power receptacle or communications interface (e.g., electronics module 325).

In various other embodiments, the detection device of the present disclosure may be associated with a software. The software be embodied in, for example, a mobile application, a web application, a server application, and/or a desktop application. Through communication means detailed at least with reference to FIG. 4 below, the software may be configured to provide and display a plurality of reports associated with the data received from the detection device. The reports may include, for example, a combination for graphical and textual elements representing data gathered from the detection device and other processed metrics. Additionally, the reports may be integrated with information from other measuring/monitoring devices to help clarify underlying causes (e.g., a Fitbit® report identifying exercise level).

The software may be further configured to send alerts (e.g., telecommunications including, but not limited to, for example, email, SMS, telephonic alerts) to selected parties. The selected parties may comprise, but not be limited to, friends and relatives, emergency contacts, or the user's physicians who have interest in the user's health condition. Through the software, a user may define a plurality of third parties that are to be contacted in the event an increased cough rate has been detected by the device. Furthermore, the user may configure a message to be communicated to those third parties along with the data.

As will be further detailed below, the software may enable a user to adjust various settings associated with the device, including, but not limited to, for example, a base-line waveform, threshold detection levels, communication rules, alert settings, permitted parties, and the like. In some embodiments, the software may be installed onto a mobile computing device (e.g., a smartphone) in operative communication with the device. In some embodiments, the software may be embodied in an apparatus such as, for example, a baby-monitoring device. The software may be operative with various elements of the mobile computing device, such as, but not limited to, microphones, global positioning elements, cell tower communication, accelerometers, internet connectivity and audio, visual, and tactile feedback mechanisms.

Still consistent with embodiments of the present disclosure, the location of the detection device may be tracked using, for example, global positioning elements of the mobile computing device in communication with the detection device. In turn, the cough detection information received with the detection device may be associated with the user's location as derived from the mobile computing device. Having the user's location, a third party who may receive an alert issued by the detection device may be better enabled to provide emergency on-site assistance to the user. Similarly, in some embodiments, a microphone of the mobile computing device may be used in conjunction with a microphone of the detection device. Employing both microphones as a source of input may lead to better cough detections.

In various embodiments, the software may further comprise an individual's personalized treatment plan, for example, a person's asthma action plan. The asthma action plan may provide helpful guidance to the user to properly interpret and act upon symptoms. Moreover, the software may be configured to display predictive analytics associated with the cough detection information. These analytics may be provided by a third-party physician, for example, or computationally derived by the external device.

Consistent with embodiments of the present disclosure, the user's physician may access the software (via, for example, the web application) to review the information received and derived from the user's detection device. In turn, the physician may diagnose and prescribe a treatment for the user. The prescribed treatment may serve as an input to the software, which, in turn, may be communicated to the user by means of, for example, the user's mobile application.

II. Operating Environment

FIG. 4 illustrates an embodiment of an operating environment 400 for the cough detection, analysis, and communications platform of the present disclosure. The platform may comprise detection devices 405. Detection devices 405 may be, for example, the cough detection devices described with reference FIG. 1-3.

Detection devices 405 may comprise a communications module operative to communicate cough detection data to a centralized server 410. The communications module may be enabled to communicate using Wi-Fi, Bluetooth, cell-tower, broadband, and other telecommunications circuitry to provide communication with centralized server 410. Centralized server 410 may be in operative communication with detection devices 405 via, for example, a telecommunications channel such as, but not limited to, for example, local area network, a wide area network, an internet connection, an/or a cloud computing environment.

Not shown in FIG. 4, detection devices 405 may be further configured to employ an intermediary channel in order to connect to server 410. For example, the communications module of detection devices 405 may only be configured for interaction in a Bluetooth or Wi-Fi communications protocol, yet, in some embodiments, centralized server 410 may not accessible through these protocols. Accordingly, detection devices 405 may employ a Wi-Fi and/or Bluetooth enabled communications module to communicate data to a mobile computing device (e.g., the user's smartphone or other bridge device with limited functionality, including, but no limited to, for example, Bluetooth, cellular radio, GPS, and power supply). The mobile computing device may have mobile application of the platform the installed thereon. In turn, the mobile computing device may be configured communicate the data received from the detection device to centralized server 410, receive data from centralized server 410, and provide data back to the detection device 405.

Server 410 may be operative to process the data received from the detection device 405 (either directly or indirectly through the mobile computing device) and communicate the processed data in accordance to the various embodiments of the present disclosure. The processed data may be received at a user-interface (UI) 415 consistent with embodiments of the present disclosure. As mentioned above, UI 415 accessible by the user of the device or a third party, such as, for example, a physician, administrator, relative, or the detection device user via, for example, a web application or a mobile application consistent with embodiments of the present disclosure.

III. Platform Operation

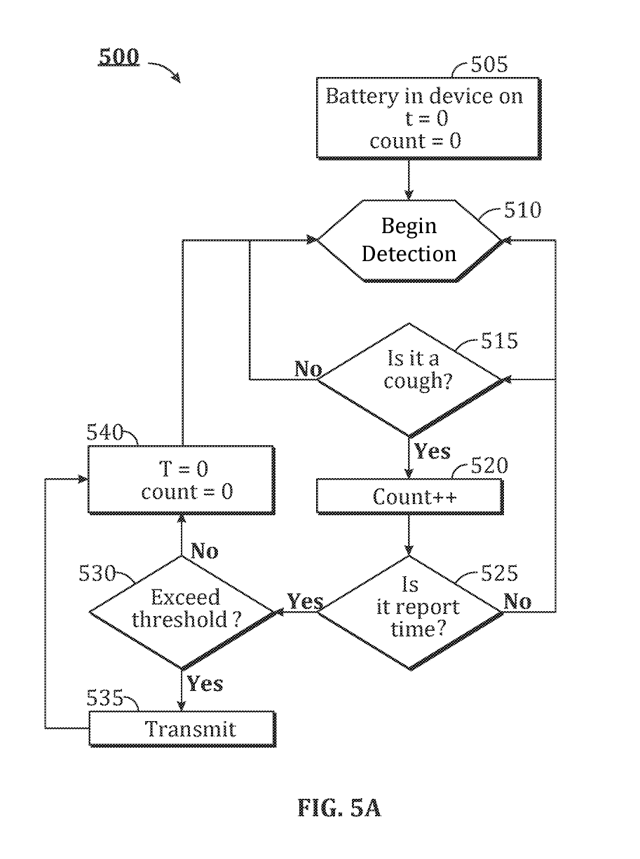

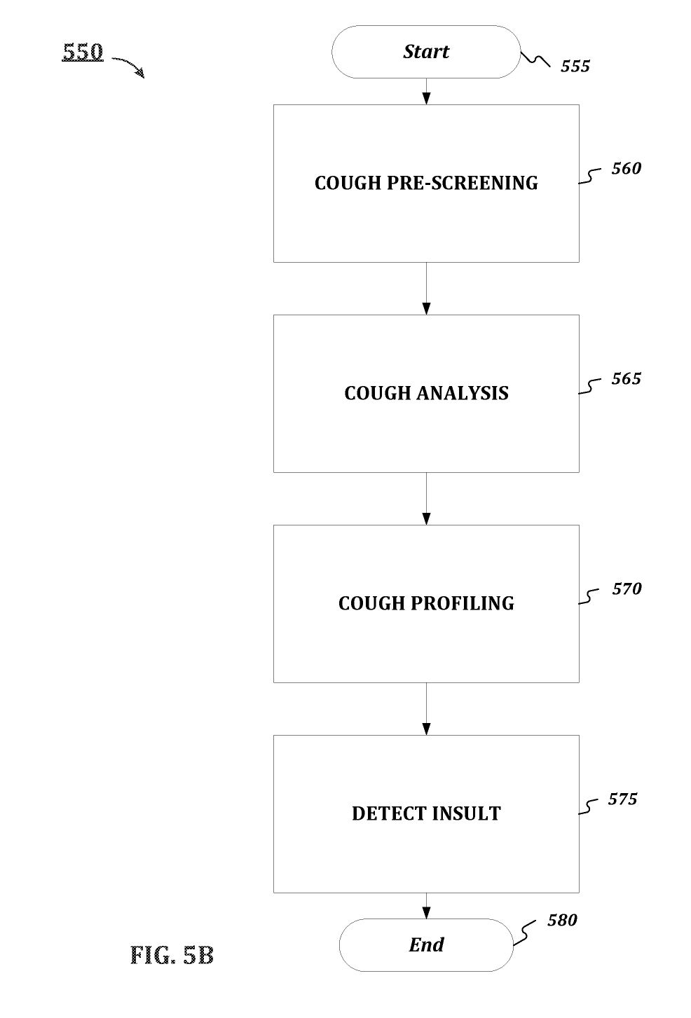

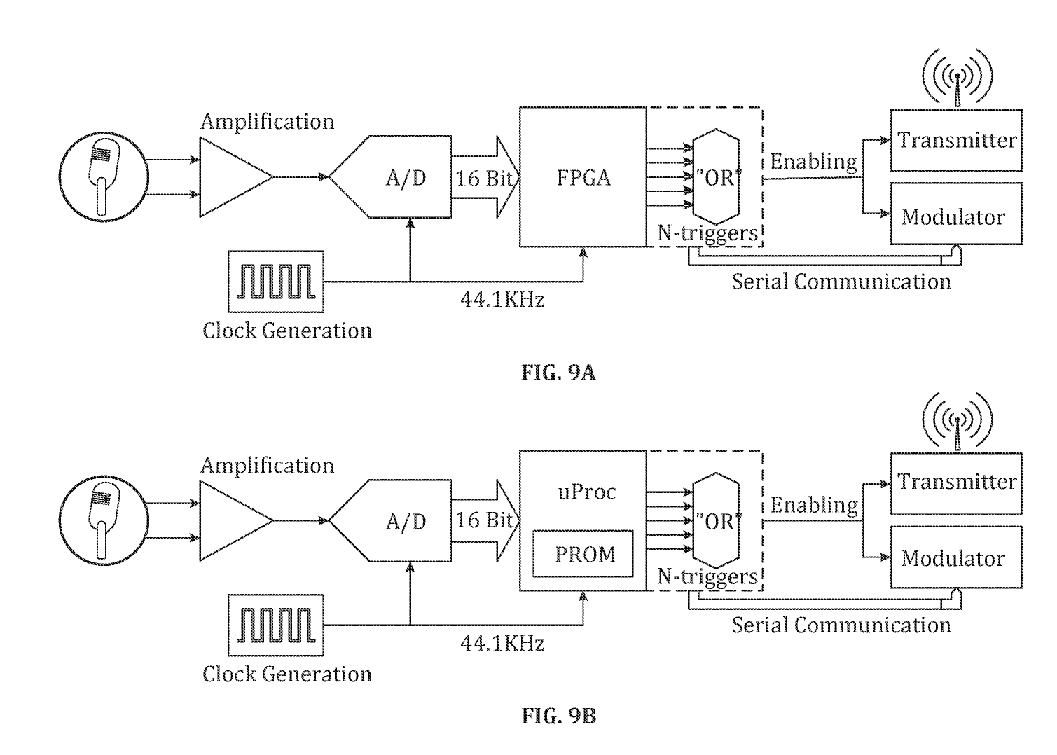

FIG. 5A is a flow chart setting forth the general stages involved in a method 500 consistent with embodiments of the disclosure for providing the cough detection, analysis, and communications platform of the present disclosure. Still consistent with embodiments of the present disclosure, method 500 may be implemented by a combination of hardware elements (e.g., counters, timers, FPGA chips, etc.) and/or software elements (e.g., smartphone application, firmware, etc.) as shown in FIGS. 9A-9B.

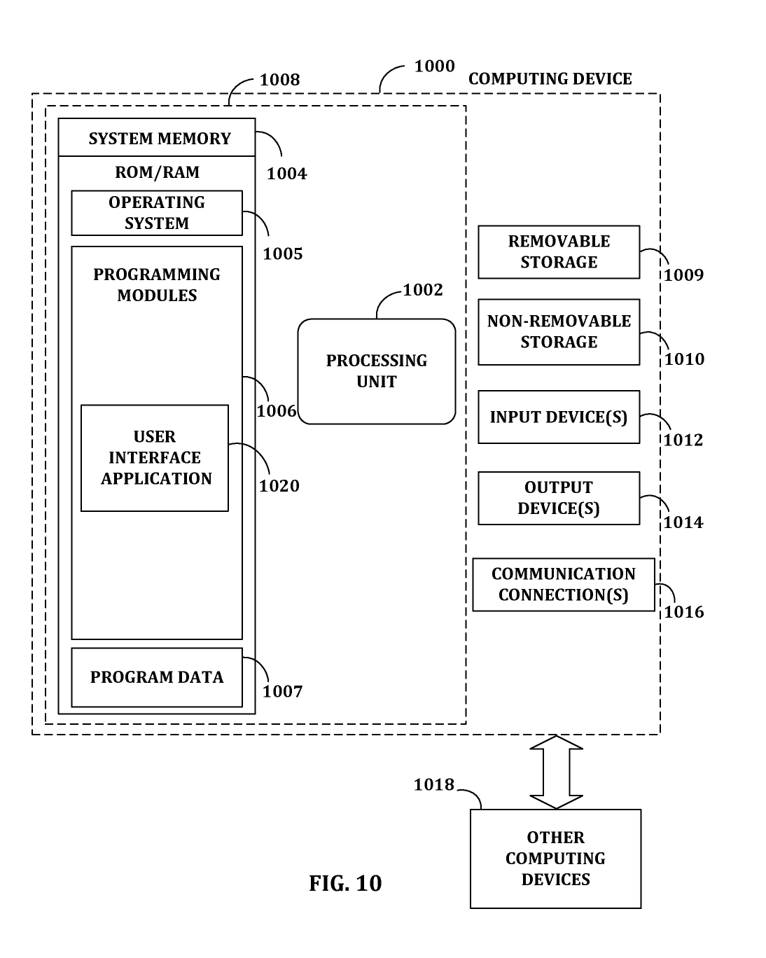

Moreover, in some embodiments, method 500 may be performed by computing device 1000 illustrated in FIG. 10. In these embodiments, detection device 405 may serve as remote microphone that transmits a signal to the computing device 1000 (e.g., a mobile computing device/users smartphone via a Bluetooth connection) which may then, in turn, perform the processing and communications of method 500. In other embodiments, a portion of the processing disclosed in method 500 may be performed by the detection device 405, while a portion of the processing may be performed by the mobile computing device or bridge device in operative communication with the detection device 405. In yet further embodiments, method 500 may be performed by device 405 in its entirety.

Though the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 500 will be described in greater detail below.

The method may begin at stage 505 where detection device 405 may be turned on. Device 405 may be turned on by activation of an on-off switch or activating a battery contact. In embodiments where the device 405 is configured to be in communication with a proximately located mobile computing device (e.g., a user's smartphone), device 405 may be registered with the mobile computing device once turned on. In certain embodiments of the present disclosure, device 405 may always be on, but running in a passive detection state.

Upon detecting a trigger in the passive detection state, device 405 may enter an active detection state. The trigger may comprise, but not be limited to, for example, a microphone pick-up of a sound exceeding a predetermined threshold. The sound may be an indication of a disease exacerbation trigger. Once triggered, device 405 may be configured to operate at the active detection state for a preset period of time.

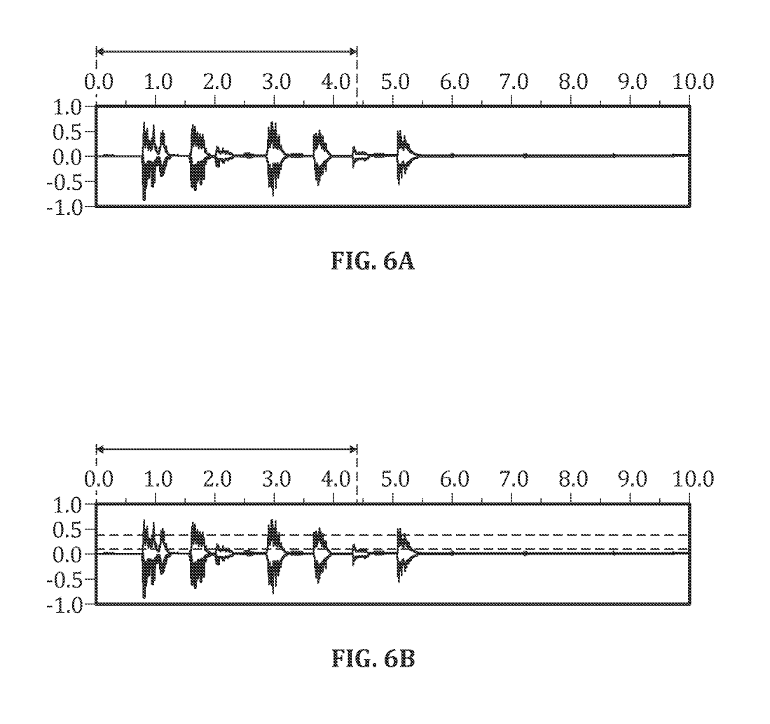

In the active detection state, a counter and a timer may be set to a value of 0, and cough detection may begin at stage 510. A microphone input of device 405 may be used to detect sounds. As will be described with greater detail below with reference to FIGS. 6-8, a software or firmware running on the device may employ various cough detection algorithms to distinguish coughs from other detected sounds.

Once a sound exceeding the threshold is detected, device 405 may proceed to stage 515 where it may employ the cough detection algorithms to determine whether the detected sound comprises a cough. The cough detection algorithms may be further configured to ascertain whether the detected sound is likely to be a cough. Although an exact detection may not always be made, an approximation or estimate may be still be useful in some applications of the present disclosure (e.g., where the coughing rate is so high that misrecognition may be a non-issue).

If it is determined that the detected sound is not a cough, device 405 may return to detection stage 510. If it is determined that the detected sound is a cough, device 405 may proceed to stage 120 where it may register the cough by incrementing a counter.

From stage 520 where the counter is incremented, device 405 may advance to stage 525 where it checks the timer to determine if the predetermined period of time has expired. If detection time has not expired, the device returns to detection stage 510, and the timer continues to run while the counter continues to be incremented for each detected cough. If detection time has expired, device 405 may proceed to stage 530 where it determines whether the cough counter has exceeded a preset threshold. If the cough counter has exceeded the threshold, device 405 may proceed to stage 535 where it transmits the cough count to server 410.

Consistent with various embodiments, device 405 may be configured to transmit other data in addition to the cough count, such as, but not limited to, historical count information, time information, and location information. As will be detailed below, the transmitted data may comprise a waveform of the detected sounds or an interpretation of the waveform. Moreover, in embodiments where device 405 is configured with other health monitoring utilities, such as heart rate, blood pressure, and blood sugar levels, the device may be operative to transmit corresponding readings with those utilities as well.

As mentioned above, device 405 may communicate with server 410 via an intermediary channel provided by a mobile computing device in communication with device 405. For example, device 405 may provide data to the mobile computing device, which may then, in turn, relay the data to a cloud-based server or third parties defined by the user. In various other embodiments, device 405 may be operative to transmit data directly to server 410.

Once the information is transmitted, device 405 may advance to stage 540 where it may reset the timer and cough counter, and proceed to stage 510 where it may continue in the active detection state for another cycle. In some embodiments, the cough counter may not be reset, and the threshold in stage 530 may be compared to a second threshold corresponding to a two period cycle of detection.

Consistent with embodiments of the present disclosure, different cough count and time combinations may correspond to different signals being transmitted by device 405. For example, if a first quantity of coughs were detected in a first cycle (e.g., six coughs were detected in the first fifteen minute cycle) and another quantity of coughs were detected in the second cycle (e.g., six coughs were detected in a second fifteen minute cycle), a first signal may be transmitted. Similarly, if, for example, ten coughs were detected in the first fifteen minute cycle and another fifteen coughs were detected in the second fifteen minute cycle, a second signal may be transmitted.

In this way, different cough rates may be configured to cause a transmission of different signals. The signals may be preset signals which may, in turn, each trigger different actions at by server 410 or a mobile computing device in operative communication with device 405. Moreover, because signal transmission may substantially impact battery drainage, a power management configuration may cause the transmission circuitry of the detection device to turn on in periodic short bursts as needed.

If in stage 530 it is determined that cough counter has not exceeded the threshold, then detection device may proceed to stage 540, reset the timer and cough counter, and enter into the passive detection state. When the device enters back into the passive state, an “all clear” signal may be transmitted. Additional information, such as a low batter warning, may be transmitted when necessary.

FIG. 5B is a flow chart setting forth the general stages involved in a method 550 consistent with an embodiment of the disclosure for performing cough detection. FIGS. 6-9 further detail the signal processing and communications aspects disclosed by method 550.

Although method 550 has been described to be performed in environment 400 in conjunction with at least one of detection device 405 and server 410, it should be understood that, in some embodiments, different operations may be performed by different environment elements. For example, server 410 (as further detailed with respect to computing device 1000) may be employed in the performance of some or all of the stages in method 550. It should be understood that either detection device 405 or server 410 may be employed in the performance of some or all of the stages in method 550.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method @00 will be described in greater detail below.

Method 550 may begin at starting block 555 and proceed to stage 560 where a detected sound may be pre-screened for a cough. Screening may be performed to detect a cough-like characteristic of individual detected sounds. For example, using sound energy over time (as further detailed in the figures below), a cough may be detected. By way of non-limiting example, detecting energy over a long duration of time may be a cough, whereas a door slam may be a detected energy over a short period of time).

From stage 560, method 550 may advance to stage 565 where the pre-screened detected cough is further analyzed. Further analysis may comprise, for example, a comparison of the sound waveform profile (e.g., shape and duration compared against a pre-set baseline waveform known to be a cough) associated with the detected cough, along with an analysis of appropriate detailed waveform characteristics in low, middle and high frequency sound ranges and waveform shape (beginning, middle, and end).

Various embodiments of the present disclosure may use the large energy in the low frequency 20-320 Hz range to profile productive cough versus unproductive cough (in COPD, unproductive cough may be correlated with a patient's background chronic cough). Accordingly, embodiments may also be configured to determine background chronic cough baseline much more simply by counting the patient cough frequency in between the 25 cough/hour episodes).

Method 550 may continue to stage 570 where the detected sound may be profiled as a cough sound for disease appropriateness to score the cough episode for sending the appropriate alert. By way of non-limiting example, the following factors may be considered in profiling a cough to issue an alert:
Cough frequency: asthma may be 50 coughs per hour, COPD may be 25 coughs per hour, and CF may be 110 coughs per hour, and
Duration of increased cough frequency (e.g., asthma is 10 minutes or more, respiratory infection is 2 minutes or less), and
Overall waveform characteristic (e.g., asthma cough waveform may be a two-humped camel with a long tail; pneumonia cough waveform may be a one-humped camel with a short tail, TB may be a three humped camel with a short tail and the middle hump may be a spiked waveform representing the TB cough cackle, etc.)

Embodiments may perform this overall waveform analysis from the analysis in stage 565 and from comparing the waveform shape to a library of well characterized cough waveforms.

Furthermore, in some embodiments, an additional analysis of the detected sound may be performed in order to individualize the detection since the overall energy of the cough as well as the frequency range (overall lower or higher frequency of cough waveform) can be used to associate a cough sound to an individual. This may be appropriate for determining the cough is from one person versus another where two people coughing are near each other.

Method 550 may proceed to stage 575 where the cough frequency profile may be finalized as an insult that may lead to an alert triggering event (e.g., an asthma attack, respiratory infection, or a baseline unproductive coughing). Method 550 may then end at stage 580.

IV. Signal Processing

The creation and manufacture of a low cost wearable device to detect human coughing provides challenges in as much as the human cough is not closely modeled by a sonic impulse but rather a time extended ‘chirp’ of complex frequencies that can last for approximately 200 ms to 400 ms. FIGS. 6-8 illustrate charts corresponding cough detection algorithms that may be used with embodiments of the present disclosure. Embodiments of the present disclosure may employ, for example, two or more microphones embedded within detection device 405.

The signal illustrated in FIG. 6A represents a subject who made a distinct effort to cough five times over a period of approximately five seconds. Even though the subject attempted to make the five coughs as distinct as possible, a natural ‘bounce’ occurred in the subject's chest cavity that caused secondary trailing sounds.

The signal in FIG. 6A comprises five major ‘chirps’ (or regions) corresponding to five detected coughs. The first region comprises a set of two or three distinct sounds, while the second and forth coughs are followed by noise representing the subject's chest bounce. Each person may have his/her own specific sequence of cough/transient waveforms that may vary with, for example, a type, severity and location of lung distress.

FIG. 6B illustrates a first filter that may be applied to the sound to better distinguish coughs from noise (e.g. the subsequent chest bouncing after the cough). The horizontal lines added represent ‘threshold’ references (e.g. amplitude threshold slicing points) that correspond to a y-axis band-pass type filter for the amplitude of the waveform.

As can be understood form FIG. 6B, the threshold slicing points would render different cough counts depending upon their position on the waveform. For example, an overly low threshold may allow ambient noise to pass through the filter and render the cough counting less effective. On the other hand, an overly high threshold may miss coughs and, consequently, give false counts.

A cough detection device consistent with embodiments of the present disclosure may employ this filter type in cough detection. Threshold slicing may be calibrated, employ additional algorithms, and vary from embodiment to embodiment of the present disclosure. Nevertheless, no matter what threshold values may be set for this filter, there may be an error as large as 50% for time domain detection methods (TDDM). Accordingly, embodiments of the present disclosure that employ this filter type may trigger (i.e. provide a warning notification) on a ‘loose’ cough count target over an extended period of time.

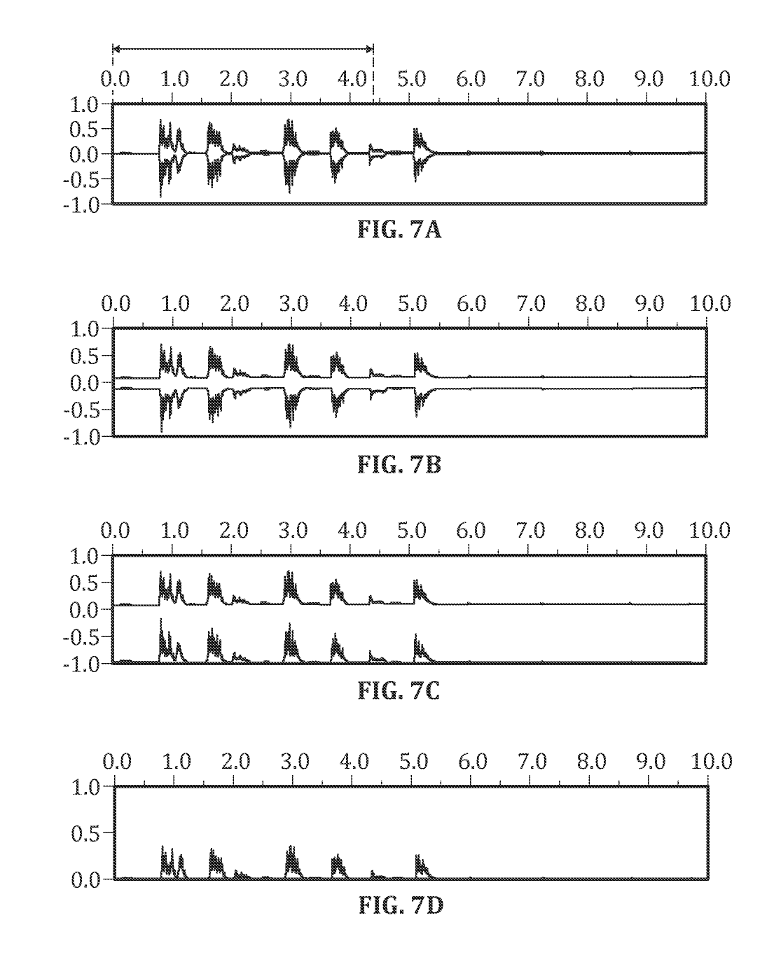

Various embodiments of the present disclosure may also employ frequency based methods of cough detection and counting. FIG. 7A illustrates the signal of a subject's five cough cycle. The signal may be captured by an AC coupled (DC removed) microphone and amplified by methods available to one of ordinary skill in the art.

Using a 0V threshold, the signal may be split into positive and negative going voltages as illustrated in FIG. 7B. The upper and lower signal portions may be vary slightly, so both portions should be used in the detection. As illustrated in FIG. 7C, the upper voltage may be preserved as V_{after}+=V_{before}+, while the lower waveform may be inverted as V_{after}−=−(V_{before}−).

This signal portions of FIG. 7C may then be passed to an Operational Amplifier to subtract the two portions, yielding Vᵣₑₛᵤₗₜ=V_{after}+−V_{after}−. Vᵣₑₛᵤₗₜ, depicted in FIG. 7D, may represent the absolute value of the signal illustrated in FIG. 7A.

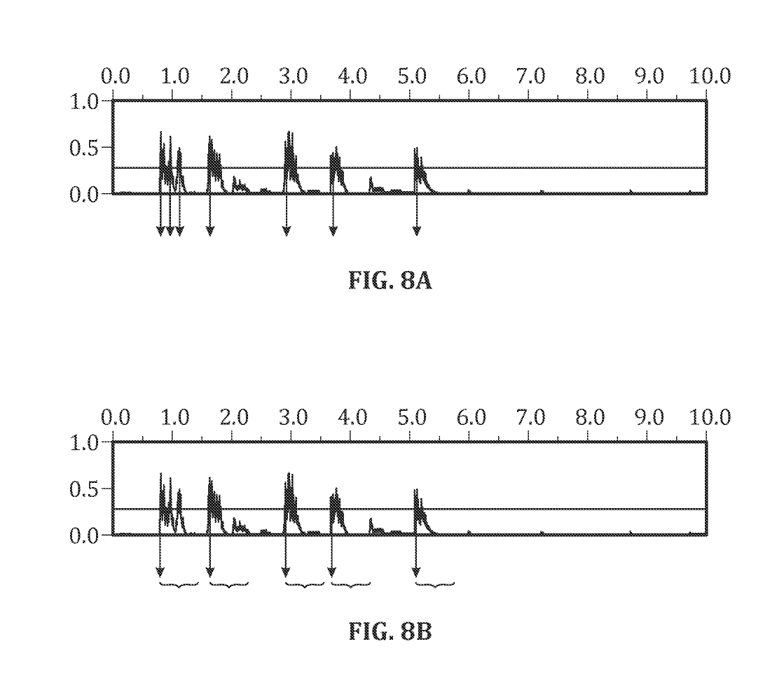

The Signal-to-Noise Ratio (SNR) of the final waveform in FIG. 7D may be improved for better cough detection. For example, applying a straight threshold with a rising edge trigger for counting coughs like the one shown in FIG. 8A, seven coughs may be detected. This may result in an error of 40% since the subject only coughed five times. Knowing that distinct coughs may not occur in intervals of less than, for example, 300 ms, the detection error may be improved. Consistent with various embodiments of the present disclosure, a disable circuit may be activated upon each rising edge trigger. The disable circuit would effectively prevent or hold the rising edge trigger from activating for at least a predetermined interval of, for example, 300 ms. In this way, the cough count may be incremented by rising edge triggers is not activated for the noise occurring in between the actual coughs. FIG. 8B shows the rising edge trigger counting methods coupled with a disabling circuit at approximately 300 ms intervals. As a result, the rising edge trigger method may now count five coughs.

Embodiments of the present disclosure may further improve cough detection by personalizing the detection device to the individual cough print or waveform of the user. For example, in addition to having stored a small library of recorded coughs associated with different gender, ages, and types of coughs, a cloud-based server may be configured to receive, from the detection device, a cough of the user.

Consistent with embodiments of the present disclosure, when the detection device is initialized, a user may record a cough to the device. The device may then be configured to communicate this recorded cough to an external device, such as a smartphone app or a cloud-based server (e.g., server 410).

The external device, such as the cloud-based server, may then process the received recording and derive a baseline dataset to be used as a comparison point by the detection device. The baseline dataset may comprise, for example, a baseline waveform and/or other data to which a signal may be compared and analyzed for cough detection. In turn, the external device may communicate the baseline waveform back to the detection device. The detection device may, in turn, update its processing algorithms for cough detection based on the updated baseline dataset. In this way, if the user were to lose his detection device or otherwise replace his detection device, the algorithm update may be applied to easily personalize the detection device to the user.

Moreover, as the cough-detection algorithm improves, the software may be continuously upgraded by connection to the external device. The cough-detection algorithm may improve with use of the detection device, as it may learn and build a user-specific cough profile. In accordance to the embodiments of the present disclosure, the connection may not be limited to a wireless connection. For instance, the connection may be a wired, Universal Serial Bus (USB) connection.

V. Detection Device Architecture

By way of non-limiting example, FIGS. 9A and 9B illustrate an architecture for two embodiments of the detection device. The first embodiment may be operated by a Field-Programmable Gate Array (FPGA) chip while the second embodiment may be operated with a uProc approach. It should be understood that other architectures are possible (e.g., system on chip (SOC)). The hardware components may be operative to perform the detection, analysis and processing disclosed in the various embodiments of the present disclosure (e.g., method 500).

Consistent with embodiments of the present disclosure, an AC small amplitude response may be received by a microphone integrated in the detection device. In some embodiments, two or more microphones may be used (e.g., one on each side of the detection device). Using a weak microphone pendent resting on a user's chest cavity, rather than the sensitive microphone, may facilitate the filtering of non-cough related sounds as distant sounds may not be detected by a weak microphone.

The signal from the microphone may then be passed to a differential amplifier for a single ended output to an analog/digital (A/D) converter. The A/D converter may output a 16 bit word as clocked by clock generation circuitry coupled to the converter.

The clock generation circuitry may produce a clock sample rate (Fs) of 44.1 KHz, corresponding to the Nyquist frequency of the human voice. Accordingly, this Fs may eliminate aliasing of the human voice. Lower sampling rates may be used for cough detection. The clock generation circuitry may also clock the FPGA, uProc, or any other programmable microprocessor with onboard memory (hereinafter “processing unit”) that may be designed with the detection device.

The processing unit may have programmed, in its associated memory storage, computer readable code operative to perform methods of FIGS. 5A and 5B. For instance, the N-Triggers associated with the processing unit may produce HIGH true signals whenever a given threshold of coughs over a period time is exceeded. The N-Triggers may form a parallel input “OR” which produces an enabling signal that may be passed to the transmitter and modulator.

The enabling signal may enable the transmitter and modulator to power up. The serial stream received from the processing unit may be modulated by the modulator and then broadcast over the transmitter. The serial stream may convey source, destination, and detection information to the modulator and, in turn, the transmitter.

After transmission, the detection device may power down the transmitter and the modulator and reinitialize itself. Generally, consistent with embodiments of the disclosure, the detection device may comprise program modules that may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems. Embodiments of the disclosure, for example, may be implemented as a computer process (method), a computing system, or as an article of manufacture, such as a computer program product or computer readable media.

The computer program product may be a computer storage media readable by a computer system and encoding a computer program of instructions for executing a computer process (e.g., method 500). The computer program product may also be a propagated signal on a carrier readable by a computing system and encoding a computer program of instructions for executing a computer process.

Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable readable medium may include the following: an electrical connection having one or more wires, a random access memory (RAM), a read-only memory (ROM), an erasable programmable read-only memory (EPROM or Flash memory), an optical fiber, and the like. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

VI. Platform Architecture

The platform of the present disclosure may be embodied as, for example, but not be limited to, a website, a web application, a desktop application, and a mobile application compatible with a computing device. The computing device may comprise, but not be limited to, a desktop computer, laptop, a tablet, mobile telecommunications device, or a detected local gateway to telecommunications network. Moreover, the platform may be hosted on a centralized server (e.g., 410), such as, for example, a cloud computing service.

Embodiments of the present disclosure may comprise a system having a memory storage and a processing unit. The processing unit coupled to the memory storage, wherein the processing unit is configured to perform at least a portion of the stages of methods 500 and 550.

FIG. 10 is a block diagram of a system including computing device 1000. Consistent with embodiments of the disclosure, the aforementioned memory storage and processing unit may be implemented in a computing device, such as computing device 1000 of FIG. 10. Any suitable combination of hardware, software, or firmware may be used to implement the memory storage and processing unit. For example, the memory storage and processing unit may be implemented with computing device 1000 or any of other computing devices 1018, in combination with computing device 1000. The aforementioned system, device, and processors are examples and other systems, devices, and processors may comprise the aforementioned memory storage and processing unit, consistent with embodiments of the disclosure.

With reference to FIG. 10, a system consistent with embodiments of the disclosure may include a computing device, such as computing device 1000. In a basic configuration, computing device 1000 may include at least one processing unit 1002 and a system memory 1004. Depending on the configuration and type of computing device, system memory 1004 may comprise, but is not limited to, volatile (e.g. random access memory (RAM)), non-volatile (e.g. read-only memory (ROM)), flash memory, or any combination. System memory 1004 may include operating system 1005, one or more programming modules 1006, and may include a program data 1007. Operating system 1005, for example, may be suitable for controlling computing device 1000′s operation. In one embodiment, programming modules 1006 may include for example, user interface application 1020. User interface application 1020 may be operative to provide and/or communicate the data necessary to cause a display and corresponding functionality of a user interface on a mobile application or web application associated with the platform. Furthermore, embodiments of the disclosure may be practiced in conjunction with a graphics library, other operating systems, or any other application program and is not limited to any particular application or system. This basic configuration is illustrated in FIG. 10 by those components within a dashed line 1008.

Computing device 1000 may have additional features or functionality. For example, computing device 1000 may also include additional data storage devices (removable and/or non-removable) such as, for example, magnetic disks, optical disks, USB storage, or tape. Such additional storage is illustrated in FIG. 10 by a removable storage 1009 and a non-removable storage 1010. Computer storage media may include volatile and nonvolatile, removable and non-removable media implemented in any method or technology for storage of information, such as computer readable instructions, data structures, program modules, or other data. System memory 1004, removable storage 1009, and non-removable storage 1010 are all computer storage media examples (i.e., memory storage.)

Computing device 1000 may also have input device(s) 1012 such as a keyboard, a mouse, a pen, a sound input device, a touch input device, etc. Output device(s) 1014 such as a display, speakers, a printer, etc. may also be included. The aforementioned devices are examples and others may be used.

Computing device 1000 may also contain a communication connection 1016 that may allow device 1000 to communicate with other computing devices 1018, such as over a network in a distributed computing environment, for example, an intranet or the Internet. Communication connection 1016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1004, including operating system 1005. While executing on processing unit 1002, programming modules 1006 (e.g. user interface application 1020) may perform processes associated with the enablement of the mobile and web applications of the platform. The aforementioned process is an example, and processing unit 1002 may perform other processes. Other programming modules that may be used in accordance with embodiments of the present invention may include electronic mail and contacts applications, word processing applications, spreadsheet applications, database applications, slide presentation applications, drawing or computer-aided application programs, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer storage, a random access memory (RAM), a read-only memory (ROM), an erasable programmable read-only memory (EPROM or Flash memory), an optical fiber, and the like. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, USB storage devices, or a CD-ROM, a carrier wave from the Internet, or other forms of RAM or ROM. Further, the disclosed methods' stages may be modified in any manner, including by reordering stages and/or inserting or deleting stages, without departing from the disclosure.

VII. Advantages

Embodiments of the present disclosure could assist physicians in diagnosing various diseases and disorders. For example, the cough detection analysis could aid a physician in diagnosing pulmonary conditions such as, for example, but not limited to: asthma (which is still a remarkably difficult condition to diagnose objectively both in children and adults), COPD and mixed asthma/COPD, GERD, lung cancer, cystic fibrosis, bronchiectasis, interstitial fibrosis, alveolitis.

Furthermore, embodiments of the present disclosure may be configured to combine cough data, GPS data, weather and pollen data (from various third party databases) in order to trace hotspots that are causing allergies. Using this aggregated data, embodiments of the disclosure may be enabled to predict the potential for future incidents based on our proprietary map of hotspots and, for example, but not limited to, current weather (e.g., wind direction and the like) at a given location. In this way, detected cough data (indicating, for example, asthma incidents).

Furthermore, embodiments of the present disclosure could assist physicians in assessing exacerbations of conditions already diagnosed such as asthma COPD and cystic fibrosis in terms of severity and cause, especially to try to distinguish whether or not microbial infection is paramount. Being able to differentiate an exacerbation of COPD caused by infection from other causes would be a significant breakthrough in objective diagnosis and could have a major impact on use of resources such as antibiotics and hospitalization, COPD being considered the third biggest fatal disease category worldwide.

Further still, embodiments of the present disclosure could assist physicians in distinguishing types of infection. Pneumonia, TB, whooping cough, which are emerging again as a significant cause of chronic cough may be difficult to diagnose from conventional the microbiological and serum testing methods.

Embodiments of the present disclosure may assist a physician in treating patients with cardiac based disease by, for example, facilitating the detection of coughing sounds associated with left ventricular failure or right ventricular failure and detecting rate disturbance such as paroxysmal atrial fibrillation.

VIII. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. An apparatus comprising:
at least one microphone;
a communications module; and
a processing unit associated with the microphone, the communications module, and the memory storage, wherein the processing unit is operative to:
receive a signal from the at least one microphone,
determine whether the received signal comprises a cough, and
transmit, via the communications module, data associated with the received signal.

2. The apparatus of claim 1, wherein the processing unit is further configured to establish a baseline waveform to be employed in an analysis of signals received from the at least one microphone.

3. The apparatus of claim 2, wherein the processing unit being configured to establish the baseline waveform comprises the processing unit being configured to:
receive an initialization signal from the microphone, and
determine the baseline waveform based on the initialization signal.

4. The apparatus of claim 1, wherein establishing the baseline waveform comprises receiving the baseline waveform via the communications module.

5. The apparatus of claim 1, wherein the communications module is operative to receive the signal from the at least one microphone.

6. The apparatus of claim 1, wherein the communications module is operative to receive data from a server, the received data enabling the processing unit to adjust at least one setting of the apparatus.

7. The apparatus of claim 1, wherein the processing unit is operative to receive the signal from the microphone in a passive detection state and, in response to the signal, enter into an active detection state.

8. The apparatus of claim 7, wherein the processing unit is operative to, in the active detection state:
initialize a timer set for the predetermined period of time, and initialize a counter.

9. The apparatus of claim 8, wherein the processing unit is further operative to increment the counter in response to a determination the signal received from the microphone comprises the cough.

10. The apparatus of claim 9, wherein the processing unit being operative to transmit the data comprises the processing unit being operative to transmit the data upon a threshold count being exceeded by the counter and a threshold time being exceeded by the timer.

11. The apparatus of claim 1, wherein the apparatus comprises an adhesive sleeve.

12. The apparatus of claim 11, wherein the adhesive sleeve is configured to house electronic components associated with the at least one microphone, the communications module, and the processing unit.

13. The apparatus of claim 1, wherein the adhesive sleeve is configured to secure an antenna of the communications module around a circumference of the adhesive sleeve.

14. The apparatus of claim 11, wherein the adhesive patch is configured to adhere to a user's skin.

15. A system comprising:
a cough detection device comprising:
at least one microphone,
a first processing unit configured to:
process sounds detected by the microphone, and
derive cough detection data based of the detected sounds,
a memory configured to store cough data; and
a transmitter configured to transmit the cough detection data; and
a server in operative communication with the cough detection device,
wherein the server comprises:
a memory storage, and
a second processing unit coupled to the memory storage, wherein the second processing unit is configured to:
receive the cough detection data,
associate the cough detection data with a user profile,
process the cough detection data, wherein the second processing unit being configured to process the cough detect data comprises the second processing unit being configured to:
create at least one report associated with the cough detection data, and
issue an alert to a third party associated with the user profile, and
provide a user interface for the processed cough detection data, the user interface being accessible via at least one of the following:
a mobile application, and
a web application.

16. A method comprising:
receiving a signal comprising a waveform;
screening the waveform to detect a cough-like characteristics;
upon detection of the cough-like characteristics, analyzing the waveform to detect a cough;
determining a cough-type based on the analysis; and
issuing an alert based on the cough-type.

17. The method of claim 16, wherein screening comprising analyzing the waveform's energy over a period of time.

18. The method of claim 16, wherein analyzing the waveform comprises comparing the waveform to a baseline waveform.

19. The method of claim 16, wherein determining the cough-type comprises determining the cough-type based on at least one of the following: cough frequency, duration of increased cough frequency, and overall waveform characteristic.

20. The method of claim 16, further comprising associating the detected cough with an individual.
